(19) United States
(12) Patent Application Publication
(10) Pub. No.: US 2005/0193503 A1
(43) Pub. Date: Sep. 8, 2005

(54) Composition for the oxidation dyeing of keratinous fibres containing a laccase and dyeing method using this composition

(75) Inventors: Gerard Lang, Saint Prix (FR); Jean Cotteret, Verneuil/Seine (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'OREAL S.A.

(21) Appl. No.: 11/060,579

(22) Filed: Feb. 18, 2005

Related U.S. Application Data
(63) Continuation of application No. 09/600,129, filed on Sep. 11, 2000, now abandoned.

(30) Foreign Application Priority Data
Jan. 13, 1998 (FR) 98/00,248
Dec. 16, 1998 (WO) PCT/FR98/02752

(51) Int. Cl. A61K 7/13

(52) U.S. Cl. 8/405

(57) ABSTRACT

The invention concerns a ready-to-use oxidation dyeing composition for keratinous fibres, and in particular human keratinous fibres such as hair comprising, in an a medium suitable for dyeing, at least one oxidation colouring agent, at least one cationic direct colouring agent and at least an enzyme such as laccase, as well as the dyeing method using said composition.

The subject of the invention is a composition for the oxidation dyeing of keratinous fibres, and in particular of human keratinous fibres such as hair, comprising, in a medium appropriate for dyeing, at least one oxidation dye, at least one cationic direct dye and at least one enzyme of the laccase type, as well as the dyeing method using this composition.

It is known to dye keratinous fibres, and in particular human hair, with dyeing compositions containing oxidation dye precursors, in particular ortho- and para-phenylenediamines, ortho- or para-aminophenols, heterocyclic bases generally called oxidation bases. The oxidation dye precursors, or oxidation bases, are colourless or weakly coloured compounds which, combined with oxidizing products, can give rise to dye and coloured compounds by a process of oxidative condensation.

It is also known that the shades obtained with these oxidation bases can be varied by combining them with couplers or colour modifiers, the latter being chosen in particular from aromatic meta-diamines, meta-aminophenols, meta-diphenols and certain heterocyclic compounds.

The variety of molecules used in oxidation bases and couplers allows a rich palette of colours to be obtained.

It is also known that to further vary the shades obtained and to provide them with shimmer, it is possible to use, in combination with oxidation bases and couplers, direct dyes, that is to say coloured substances which provide a colour in the absence of oxidizing agent.

The vast majority of these direct dyes belong to the family of nitro compounds of the benzene series and have the disadvantage, when they are incorporated into dyeing compositions, of leading to colours with insufficient fastness, in particular towards shampoos.

The so-called “permanent” colour obtained by means of these oxidation dyes should moreover satisfy a number of requirements. Thus, it should have no drawbacks from the toxicological point of view, it should make it possible to obtain shades of the desired intensity and it should exhibit good resistance towards external agents (light, adverse weather conditions, washing, permanent waving, perspiration, rubbing).

The dyes should also make it possible to cover grey hair, and thus should be the least selective possible, that is to say they should make it possible to obtain the smallest possible differences in colour all along the same keratinous fibre, which may indeed be differently sensitized (i.e. damaged) between its tip and its root.

The oxidation dyeing of keratinous fibres is generally carried out in an alkaline medium, in the presence of hydrogen peroxide. However, the use of alkaline media in the presence of hydrogen peroxide has the disadvantage of causing substantial degradation of the fibres, as well as decolouring of the keratinous fibres which is not always desirable.

The oxidation dyeing of keratinous fibres can also be carried out with the aid of: oxidizing systems different from hydrogen peroxide such as enzymatic systems. Thus, it has already been proposed in Patent U.S. Pat. No. 3,251,742, Patent Applications FR-A-2,112,549, FR-A-2,694,018, EP-A-0,504,005, WO95/07988, WO95/33836, WO95/33837, WO96/00290, WO97/19998 and WO97/19999 to dye keratinous fibres with compositions comprising at least one oxidation dye in combination with enzymes of the laccase type, the said compositions being brought into contact with atmospheric oxygen. These dyeing formulations, although used under conditions which do not cause degradation of the keratinous fibres comparable to that caused by dyeings carried out in the presence of hydrogen peroxide, lead to colours which are still inadequate both from the point of view of homogeneity of the colour distributed along the fibre (“unison”), from the point of view of chromaticity (luminosity) and of the dyeing power.

The aim of the present invention is to solve the problems mentioned above.

The applicant has now just discovered that it is possible to obtain novel dyes which are capable of giving intense and chromatic colours, without causing significant degradation of the keratinous fibres, which are not very selective and which are quite resistant to various attacks to which the fibres may be subjected, by combining at least one oxidation dye, at least one cationic direct dye and at least one enzyme of the laccase type.

This discovery forms the basis of the present invention.

The first subject of the invention is therefore a ready-to-use composition for the oxidation dyeing of keratinous fibres, and in particular human keratinous fibres such as hair, characterized in that it comprises, in a medium appropriate for dyeing:
at least one oxidation dye,
at least one cationic direct dye,
at least one enzyme of the laccase type.

The ready-to-use dyeing composition in accordance with the invention gives intense and chromatic colours which exhibit low selectivity and excellent properties of resistance both to atmospheric agents such as light and adverse weather conditions and to perspiration and various treatments to which the hair may be subjected (washing, permanent deformation).

The subject of the invention is also a method for the oxidation dyeing of keratinous fibres using this ready-to-use dyeing composition.

The laccase(s) used in the ready-to-use dye composition in accordance with the invention may be chosen in particular from laccases of plant origin, animal origin, fungal origin (yeasts, moulds, fungi) or bacterial origin, organisms which may be of mono- or pluricellular origin. They can be obtained by biotechnology.

Among the laccases of plant origin which can be used according to the invention, there may be mentioned the laccases produced by plants which perform chlorophyll synthesis as indicated in Application FR-A-2,694,018 such as those found in the extracts of Anacardiaceae such as for example the extracts of Magnifera indica, Schinus molle or Pleiogynium timoriense, in the extracts of Podocarpaceae, Rosmarinus off., Solanum tuberosum, Iris sp., Coffea sp., Daucus carrota, Vinca minor, Persea americana, Catharenthus roseus, Musa sp., Malus pumila, Gingko biloba, Monotropa hypopithys (Indian pipe), Aesculus sp., Acer pseudoplatanus, Prunus persica, Pistacia palaestina.

Among the laccases of fungal origin optionally obtained by biotechnology which can be used according to the invention, there may be mentioned the laccase(s) derived from Polyporus versicolor, Rhizoctonia practicola and Rhus vernicifera as indicated in Applications FR-A-2,112,549 and EP-A-504005, those described in Patent Application WO95/07988, WO95/33836, WO95/33837, WO96/00290, WO97/19998 and WO97/19999, whose content is an integral part of the present description, such as for example those derived from Scytalidium, Polyporus pinsitus, Myceliophtora thermophila, Rhizoctonia solani, Pyricularia orizae, or variants thereof. There may also be mentioned those derived from Tramates versicolor, Fomes fomentarius, Chaetomium thermophile, Neurospora crassa, Coriolus versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporiodes, Cerrena unicolor, Coriolus hirsutus, Ceriporiopsis subvermispora, Coprinus cinereus, Panaeolus papilionaceus, Panaeolus sphinctrinus, Schizophyllum commune, Dichomitius squalens and variants thereof.

The laccases of fungal origin optionally obtained by biotechnology will be preferably chosen.

The enzymatic activity of the laccases of the invention which have syringaldazine among their substrates can be defined from the oxidation of syringaldazine under aerobic conditions. The lacu unit corresponds to the quantity of enzyme catalysing the conversion of 1 mmol of syringaldazine per minute at pH 5.5 at 30° C. The unit u corresponds to the quantity of enzyme producing a delta absorbance at 530 nm of 0.001 per minute using syringaldazine as substrate, at 30° C. and at pH 6.5.

The enzymatic activity of the laccases of the invention can also be defined from the oxidation of para-phenylenediamine. The lacu unit corresponds to the quantity of enzyme producing a delta absorbance at 496.5 nm of 0.001 per minute using para-phenylenediamine as substrate (64 mM) at 30° C. and at pH 5. According to the invention, it is preferable to determine the enzymatic activity in lacu units.

The quantities of laccase used in the compositions of the invention will vary according to the nature of the laccase chosen. Preferably, they will vary from 0.5 to 2000 lacu, or from 1000 to 4×10⁷ u units, or from 20 to 2×10⁶ lacu units per 100 g of composition.

The nature of the oxidation base(s) and/or of the couplers used in the ready-to-use dyeing composition is not critical.

The oxidation bases may be chosen in particular from para-phenylenediamines, double bases, para-aminophenols, ortho-aminophenols and heterocyclic oxidation bases.

Among the para-phenylenediamines which can be used as oxidation base in the dyeing composition in accordance with the invention, there may be mentioned in particular the compounds of the following formula (I) and their addition salts with an acid: 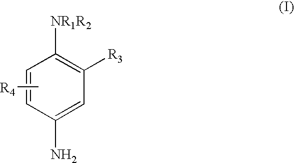
in which:
R₁ represents a hydrogen atom, a C₁-C₄ alkyl radical, a monohydroxy(C₁-C₄ alkyl) radical, a polyhydroxy-(C₂-C₄ alkyl) radical, a (C₁-C₄)alkoxy(C₁-C₄)alkyl radical, a C₁-C₄ alkyl radical substituted with a nitrogen-containing group, a phenyl radical or a 4′-aminophenyl radical;
R₂ represents a hydrogen atom, a C₁-C₄ alkyl radical, a monohydroxy(C₁-C₄ alkyl) radical, a polyhydroxy(C₂-C₄ alkyl) radical, a (C₁-C₄)alkoxy(C₁-C₄)alkyl radical or a C₁-C₄ alkyl radical substituted with a nitrogen-containing group;
R₃ represents a hydrogen atom, a halogen atom such as a chlorine, bromine, iodine or fluorine atom, a C₁-C₄ alkyl radical, a monohydroxy(C₁-C₄ alkyl) radical, a hydroxy(C₁-C₄ alkoxy) radical, an acetylamino(C₁-C₄ alkoxy) radical, a mesylamino(C₁-C₄ alkoxy) radical or a carbamoylamino(C₁-C₄ alkoxy) radical,
R₄ represents a hydrogen or halogen atom or a C₁-C₄ alkyl radical.

Among the nitrogen-containing groups of formula (I) above, there may be mentioned in particular the amino, mono(C₁-C₄)alkylamino, (C₁-C₄)dialkylamino, (C₁-C₄)trialkylamino, monohydroxy(C₁-C₄)alkylamino, imidazolinium and ammonium radicals.

Among the para-phenylenediamines of formula (I) above, there may be mentioned more particularly para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N—(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and their addition salts with an acid.

Among the para-phenylenediamines of formula (I) above, there are most particularly preferred para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, and their addition salts with an acid.

According to the invention, “double bases” is understood to mean the compounds containing at least two aromatic rings on which amino and/or hydroxyl groups are carried.

Among the double based which can be used as oxidation bases in the dyeing compositions in accordance with the invention, there may be mentioned in particular the compounds corresponding to the following formula (II), and their addition salts with an acid: 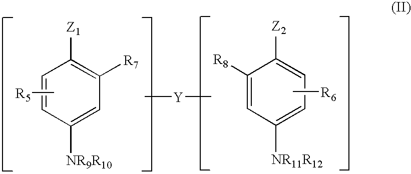
in which:
Z₁ and Z₂, which are identical or different, represent a hydroxyl or —NH₂ radical which may be substituted with a C₁-C₄ alkyl radical or with a linking arm Y;
the linking arm Y represents a linear or branched alkylene chain comprising from 1 to 14 carbon atoms, which may be interrupted by or which may end with one or more nitrogen-containing groups and/or one or more heteroatoms such as oxygen, sulphur or nitrogen atoms, and optionally substituted with one or more hydroxyl or C₁-C₆ alkoxy radicals;
R₅ and R₆ represent a hydrogen or halogen atom, a C₁-C₄ alkyl radical, a monohydroxy(C₁-C₄ alkyl) radical, a polyhydroxy(C₂-C₄ alkyl) radical, an amino(C₁-C₄ alkyl) radical or a linking arm Y;
R₇, R₈, R₉, R₁₀, R₁₁ and R₁₂, which are identical or different, represent a hydrogen atom, a linking arm Y or a C₁-C₄ alkyl radical;
   it being understood that the compounds of formula (II) contain only one linking arm Y per molecule.

Among the nitrogen-containing groups of formula (II) above, there may be mentioned in particular the amino, mono(C₁-C₄)alkylamino, (C₁-C₄) dialkylamino, (C₁-C₄)trialkylamino, monohydroxy(C₁-C₄)alkylamino, imidazolinium and ammonium radicals.

Among the double bases of formulae (II) above, there may be mentioned more particularly N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and their addition salts with an acid.

Among these double bases of formula (II), N,N′-bis(β-hydroxyethyl)-N,N′-bis(4′-aminophenyl)-1,3-diaminopropanol, 1,8-bis(2,5-diaminophenoxy)-3,5-dioxaoctane or one of their addition salts with an acid are particularly preferred.

Among the para-aminophenols which can be used as oxidation bases in the dyeing compositions in accordance with the invention, there may be mentioned in particular the compounds corresponding to the following formula (III), and their addition salts with an acid: 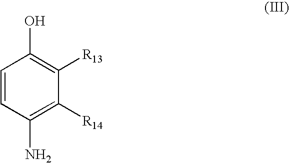
in which:
R₁₃ represents a hydrogen or halogen atom, a C₁-C₄ alkyl, monohydroxy(C₁-C₄ alkyl), (C₁-C₄)alkoxy(C₁-C₄)-alkyl, amino(C₁-C₄ alkyl) or hydroxy(C₁-C₄)alkylamino-(C₁-C₄ alkyl) radical,
R₁₄ represents a hydrogen or halogen atom, a C₁-C₄ alkyl, monohydroxy(C₁-C₄ alkyl), polyhydroxy(C₂-C₄ alkyl), amino(C₁-C₄ alkyl), cyano(C₁-C₄ alkyl) or (C₁-C₄)alkoxy(C₁-C₄)alkyl radical,
   it being understood that at least one of the radicals R₁₃ or R₁₄ represents a hydrogen atom.

Among the para-aminophenols of formula (III) above, there may be mentioned more particularly para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Among the ortho-aminophenols which can be used as oxidation bases in the dyeing compositions in accordance with the invention, there may be mentioned more particularly 2-aminophenol, 2-amino-5-methylphenol, 2-amino-6-methylphenol, 5-acetamido-2-aminophenol, and their addition salts with an acid.

Among the heterocyclic bases which can be used as oxidation bases in the dyeing compositions in accordance with the invention, there may be mentioned more particularly pyridine derivatives, pyrimidine derivatives, pyrazole derivatives, pyrazolopyrimidine derivatives, and their addition salts with an acid.

Among the pyridine derivatives, there may be mentioned more particularly the compounds described for example in Patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Among the pyrimidine derivatives, there may be mentioned more particularly the compounds described for example in German Patent DE 2,359,399 or Japanese Patents JP 88-169,571 and JP 91-333,495 or Patent Application WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and their addition salts with an acid.

Among the pyrazole derivatives, there may be mentioned more particularly the compounds described in Patents DE 3,843,892, DE 4,133,957 and Patent Applications WO 94/08969, WO 94/08970, FR-A-2,733,749 and DE 195 43 988 such as 4,5-diamino-1-methylpyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropyl-pyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylamino-pyrazole, 3,5-diamino-4-(β-hydroxyethyl)amino-1-methylpyrazole, and their addition salts with an acid.

Among the pyrazolopyrimidine derivatives, there may be mentioned more particularly the pyrazolo[1,5-a]pyrimidines of the following formula (IV), their addition salts with an acid or with a base and their tautomeric forms, when a tautomeric equilibrium exists: 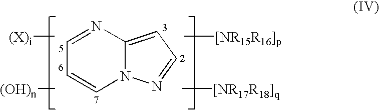
in which:
R₁₅, R₁₆, R₁₇ and R₁₈, which are identical or different, denote a hydrogen atom, a C₁-C₄ alkyl radical, an aryl radical, a C₁-C₄ hydroxyalkyl radical, a C₂-C₄ polyhydroxyalkyl radical, a (C₁-C₄)alkoxy(C₁-C₄ alkyl) radical, a C₁-C₄ aminoalkyl radical (it being possible for the amine to be protected with an acetyl, ureido or sulphonyl radical), a (C₁-C₄)alkylamino(C₁-C₄ alkyl) radical, a di-[(C₁-C₄)alkyl]amino(C₁-C₄ alkyl) radical (it being possible for the dialkyl radicals to form a carbon-containing ring or a 5- or 6-membered heterocycle), a hydroxy(C₁-C₄)alkyl- or di-[hydroxy(C₁-C₄)alkyl]-amino(C₁-C₄ alkyl) radical,
the X radicals, which are identical or different, denote a hydrogen atom, a C₁-C₄ alkyl radical, an aryl radical, a C₁-C₄ hydroxyalkyl radical, a C₂-C₄ polyhydroxyalkyl radical, a C₁-C₄ aminoalkyl radical, a (C₁-C₄)alkylamino(C₁-C₄ alkyl) radical, a di-[(C₁-C₄)alkyl]amino(C₁-C₄ alkyl) radical (it being possible for the dialkyls to form a carbon-containing ring or a 5- or 6-membered heterocycle), a hydroxy(C₁-C₄)alkyl or di-[hydroxy(C₁-C₄)alkyl]-amino(C₁-C₄ alkyl) radical, an amino radical, a (C₁-C₄)alkyl- or di-[(C₁-C₄)alkyl]-amino radical; a halogen atom, a carboxylic acid group, a sulphonic acid group;
i equals 0, 1, 2 or 3;
p equals 0 or 1;
q equals 0 or 1;
n equals 0 or 1;
   with the proviso that:
the sum p+q is different from 0;
when p+q is equal to 2, then n equals 0 and the groups NR₁₅R₁₆ and NR₁₇R₁₈ occupy positions (2,3); (5,6); (6,7); (3,5) or (3,7);
when p+q is equal to 1, then n equals 1 and the group NR₁₅R₁₆ (or NR₁₇R₁₈) and the OH group occupy positions (2,3); (5,6); (6,7); (3,5) or (3,7).

When the pyrazolo[1,5-a]pyrimidines of formula (IV) above are such that they comprise a hydroxyl group on one of the positions 2, 5 or 7 at the a position with respect to a nitrogen atom, a tautomeric equilibrium exists which is represented for example by the following scheme:

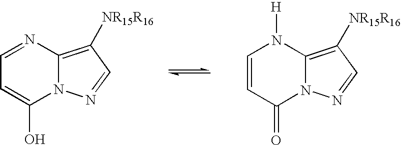

Among the pyrazolo[1,5-a]pyrimidines of formula (IV) above, there may be mentioned in particular:
pyrazolo[1,5-a]pyrimidine-3,7-diamine;
2,5-dimethylpyrazolo[1,5-a]pyrimidine-3,7-diamine;
pyrazolo[1,5-a]pyrimidine-3,5-diamine;
2,7-dimethylpyrazolo[1,5-a]pyrimidine-3,5-diamine;
3-aminopyrazolo[1,5-a]pyrimidin-7-ol;
3-aminopyrazolo[1,5-a]pyrimidin-5-ol;
2-(3-aminopyrazolo[1,5-a]pyrimidin-7-ylamino)ethanol;
2-(7-aminopyrazolo[1,5-a]pyrimidin-3-ylamino)ethanol;
2-[(3-aminopyrazolo[1,5-a]pyrimidin-7-yl)(2-hydroxy-ethyl)amino]ethanol;
2-[(7-aminopyrazolo[1,5-a]pyrimidin-3-yl)(2-hydroxy-ethyl)amino]ethanol;
5,6-dimethylpyrazolo[1,5-a]pyrimidine-3,7-diamine;
2,6-dimethylpyrazolo[1,5-a]pyrimidine-3,7-diamine;
2,5,N7,N7-tetramethylpyrazolo[1,5-a]pyrimidine-3,7-diamine;
   and their addition salts and their tautomeric forms, when a tautomeric equilibrium exists.

The pyrazolo[1,5-a]pyrimidines of formula (IV) above may be prepared by cyclization from an aminopyrazole according to the syntheses described in the following references:
EP 628559 BEIERSDORF-LILLY
R. Vishdu, H. Navedul, Indian J. Chem., 34b(6), 514, 1995.
N. S. Ibrahim, K. U. Sadek, F. A. Abdel-Al, Arch. Pharm., 320, 240, 1987.
R. H. Springer, M. B. Scholten, D. E. O'Brien, T. Novinson, J. P. Miller, R. K. Robins, J. Med. Chem., 25, 235, 1982.
T. Novinson, R. K. Robins, T. R. Matthews, J. Med. Chem., 20, 296, 1977.
U.S. Pat. No. 3,907,799 ICN PHARMACEUTICALS

The pyrazolo[1,5-a]pyrimidines of formula (IV) above can also be prepared by cyclization from hydrazine according to the syntheses described in the following references:
A. McKillop and R. J. Kobilecki, Heterocycles, 6(9), 1355, 1977.
E. Alcade, J. De Mendoza, J. M. Marcia-Marquina, C. Almera, J. Elguero, J. Heterocyclic Chem., 11(3), 423, 1974.
K. Saito, I. Hori, M. Higarashi, H. Midorikawa, Bull. Chem. Soc. Japan, 47(2), 476, 1974.

The oxidation base(s) preferably represent from 0.0005 to 12% by weight approximately of the total weight of the dyeing composition in accordance with the invention, and still more preferably from 0.005 to 6% by weight approximately of this weight.

The coupler(s) which can be used in the ready-to-use dyeing composition in accordance with the invention are those conventionally used in oxidation dyeing compositions, that is to say meta-phenylenediamines, meta-aminophenols, meta-diphenols, heterocyclic couplers, and their addition salts with an acid.

These couplers may be chosen in particular from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2,6-dimethyl-pyrazolo[1,5-b]-1,2,4-triazole, 2,6-dimethyl[3,2-c]-1,2,4-triazole, 6-methylpyrazolo[1,5-a]benzimidazole, and their addition salts with an acid.

These couplers preferably represent from 0.0001 to 10% by weight approximately of the total weight of the ready-to-use dyeing composition, and still more preferably from 0.005 to 5% by weight approximately of this weight.

The cationic direct dye(s) which can be used in the ready-to-use dyeing composition in accordance with the invention are preferably chosen from cationic aminoanthraquinones, cationic mono- or diazo compounds, cationic naphthoquinones.

By way of example, there may be used in particular [8-[(p-aminophenyl)azol]-7-hydroxy-2-napthyl]trimethylammonium chloride (also called Basic Brown 16 or Arianor Mahogany 306002 in the Color Index), 3-[(4-amino-6-bromo-5,8-dihydro-1-hydroxy-8-imino-5-oxo-2-naphthalenyl)amino]-N,N,N-trimethylbenzenaminium chloride (also called Basic Blue 99 or Arianor Steel Blue 306004 in the Color Index), 7-hydroxy-8-[(2-methoxyphenyl)azo]-N,N,N-trimethyl-2-naphthalenaminium chloride (also called Basic Red 76 or Arianor Madder Red in the Color Index), [8-[(4-amino-2-nitrophenyl)azo]-7-hydroxy-2-naphthyl]trimethylammonium chloride (also called Basic Brown 17 or Arianor Sienna Brown 306001 in the Color Index) and 3-[(4,5-dihydro-3-methyl-5-oxo-1-phenyl-1H-pyrazol-4-yl)azo]-N,N,N-trimethylbenzenaminium chloride (also called Basic Yellow 57 or Arianor Straw Yellow 306005 in the Color Index).

The cationic direct dye(s) may also be chosen from:
a) the compounds of the following formula (V): 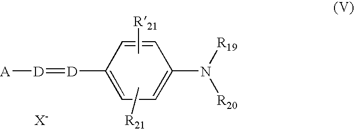
   in which:
D represents a nitrogen atom or the —CH group,
R₁₉ and R₂₀, which are identical or different, represent a hydrogen atom; a C₁-C₄ alkyl radical which may be substituted with a —CN, —OH or —NH₂ radical or form with a carbon atom of the benzene ring an optionally oxygen-containing or nitrogen-containing heterocycle which may be substituted with one or more C₁-C₄ alkyl radicals; a 4′-aminophenyl radical,
R₂₁ and R′₂₁, which are identical or different, represent a hydrogen or halogen atom chosen from chlorine, bromine, iodine and fluorine, a cyano, C₁-C₄ alkoxy or acetyloxy radical,
X⁻ represents an anion preferably chosen from chloride, methyl sulphate and acetate,
A represents a group chosen from the following structures A1 to A19: 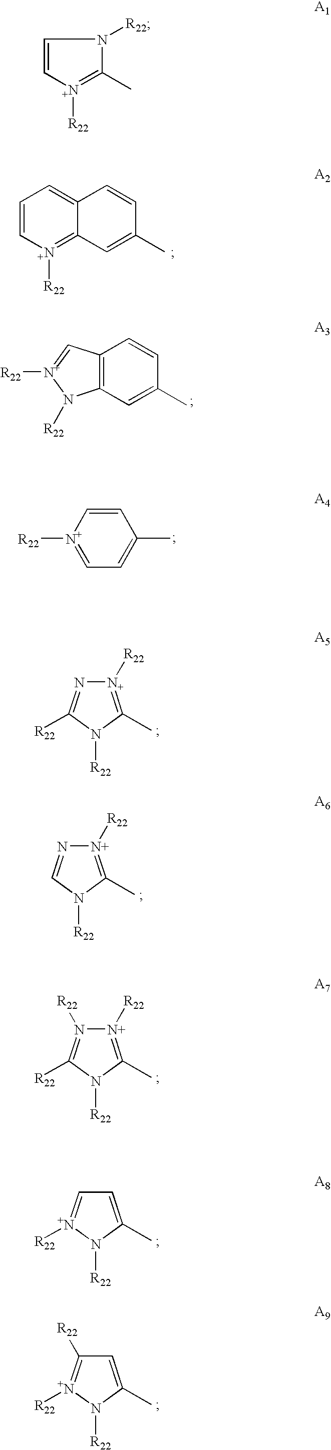 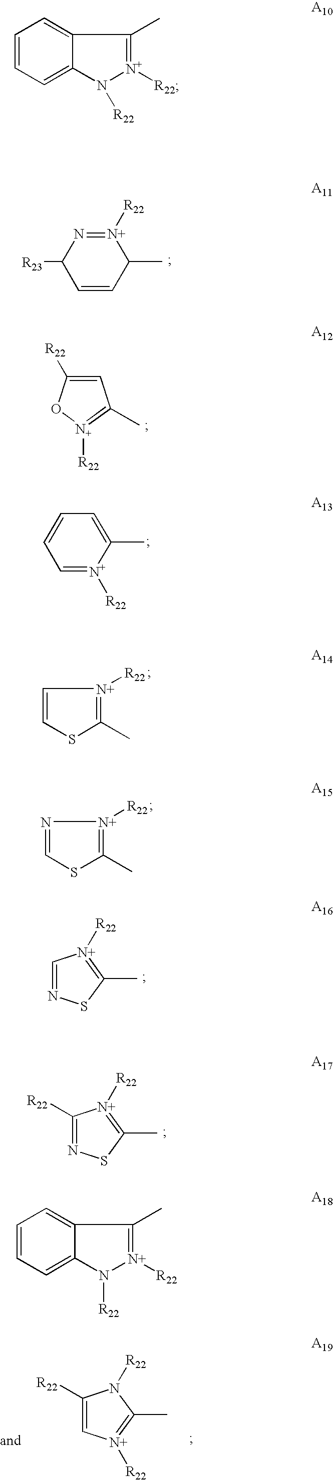
   in which R₂₂ represents a C₁-C₄ alkyl radical which may be substituted with a hydroxyl radical and R₂₃ represents a C₁-C₄ alkoxy radical;
b) the compounds of the following formula (VI): 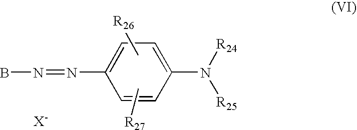
   in which:
R₂₄ represents a hydrogen atom or a C₁-C₄ alkyl radical,
R₂₅ represents a hydrogen atom, an alkyl radical which may be substituted with a —CN radical or with an amino group, a 4′-aminophenyl radical or forms with R₂₄ an optionally oxygen-containing and/or nitrogen-containing heterocycle which may be substituted with a C₁-C₄ alkyl radical,
R₂₆ and R₂₇, which are identical or different, represent a hydrogen atom, a halogen atom such as bromine, chlorine, iodine or fluorine, a C₁-C₄ alkyl or C₁-C₄ alkoxy radical, a —CN radical,
X⁻ represents an anion preferably chosen from chloride, methyl sulphate and acetate,
B represents a group chosen from the following structures B1 to B6: 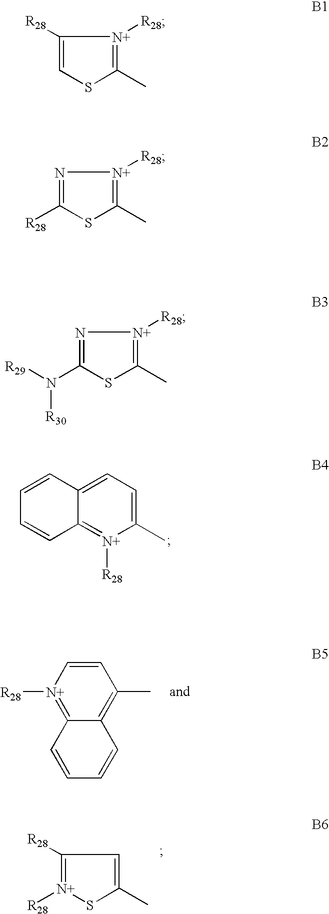
   in which R₂₈ represents a C₁-C₄ alkyl radical, and R₂₉ and R₃₀, which are identical or different, represent a hydrogen atom or a C₁-C₄ alkyl radical;
c) the compounds of the following formulae (VII) and (VII′): 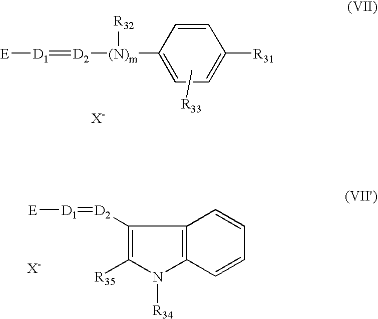
   in which:
R₃₁ represents a hydrogen atom, a C₁-C₄ alkoxy radical, a halogen atom such as bromine, chlorine, iodine or fluorine or an amino radical,
R₃₂ represents a hydrogen atom, a C₁-C₄ alkyl radical or forms with a carbon atom of the benzene ring a heterocycle optionally containing oxygen and/or substituted with one or more C₁-C₄ alkyl groups,
R₃₃ represents a hydrogen atom or a halogen atom such as bromine, chlorine, iodine or fluorine,
R₃₄ and R₃₅, which are identical or different, represent a hydrogen atom or a C₁-C₄ alkyl radical,
D₁ and D₂, which are identical or different, represent a nitrogen atom or the —CH group,
m=0 or 1,
it being understood that when R₃, represents an unsubstituted amino group, then D₁ and D₂ simultaneously represent a —CH group and m=0,
X⁻ represents an anion preferably chosen from chloride, methyl sulphate and acetate,
E represents a group chosen from the following structures E1 to E8: 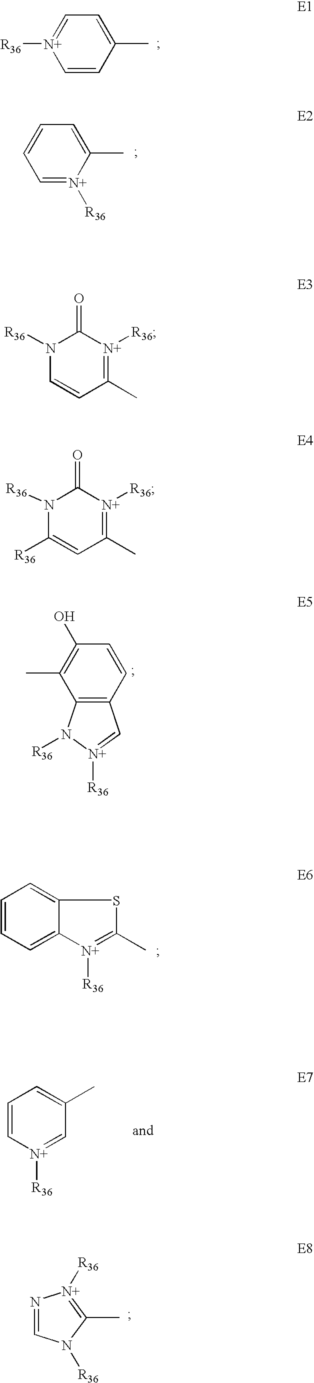
   in which R₃₆ represents a C₁-C₄ alkyl radical; when m=0 and D₁ represents a nitrogen atom, then E may also denote a group of the following structure E9: 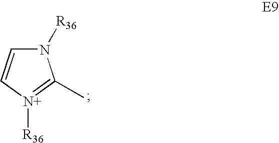
   in which R₃₆ represents a C₁-C₄ alkyl radical.

The cationic direct dyes of formulae (V), (VI), (VII) and (VII′) which can be used in the ready-to-use dyeing composition in accordance with the invention are known compounds and are described for example in Patent Applications WO 95/01772, WO 95/15144 and EP-A-0,714,954.

Among the cationic direct dyes of formula (V) which can be used in the ready-to-use dyeing compositions in accordance with the invention there may be mentioned more particularly the compounds corresponding to the following structures (V1) to (V52):

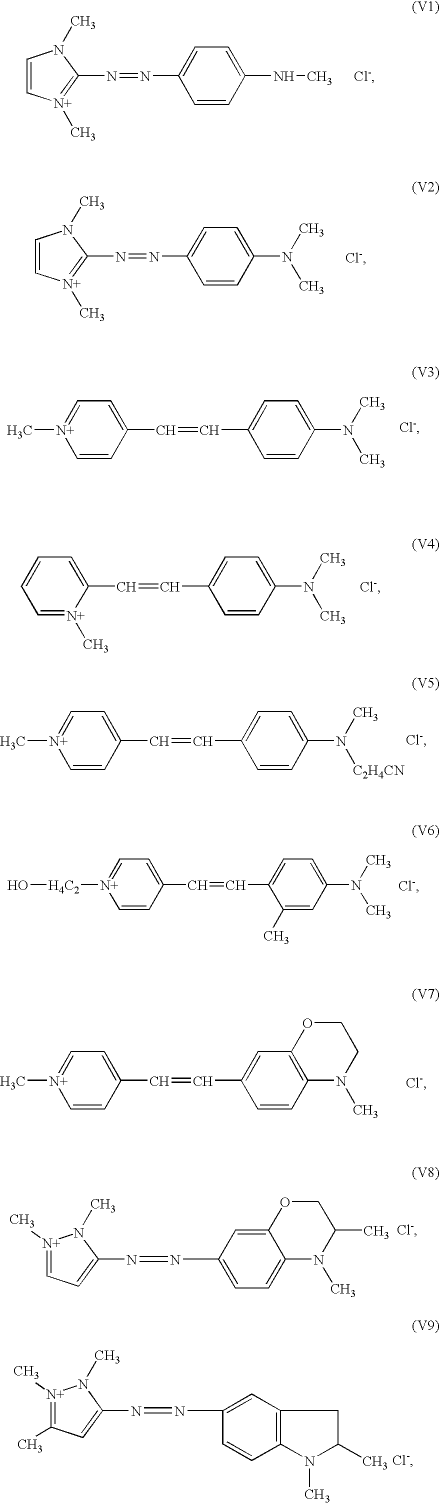

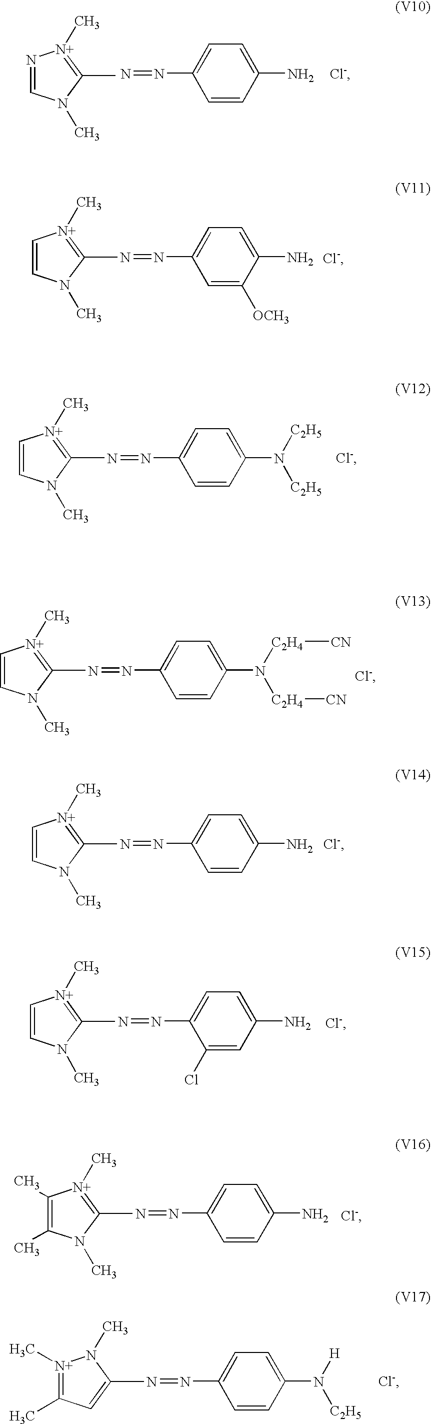

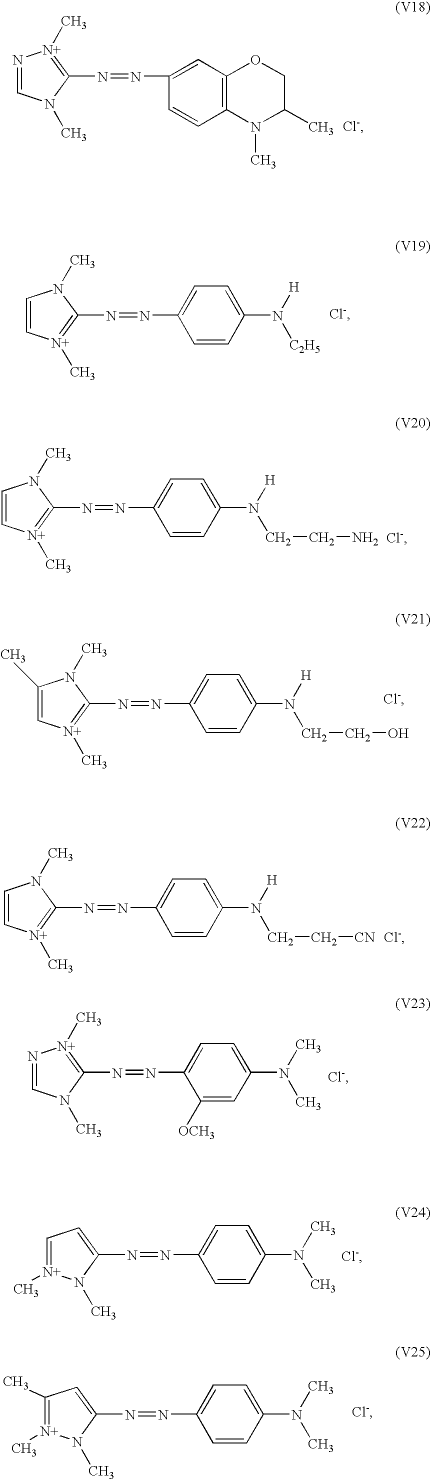

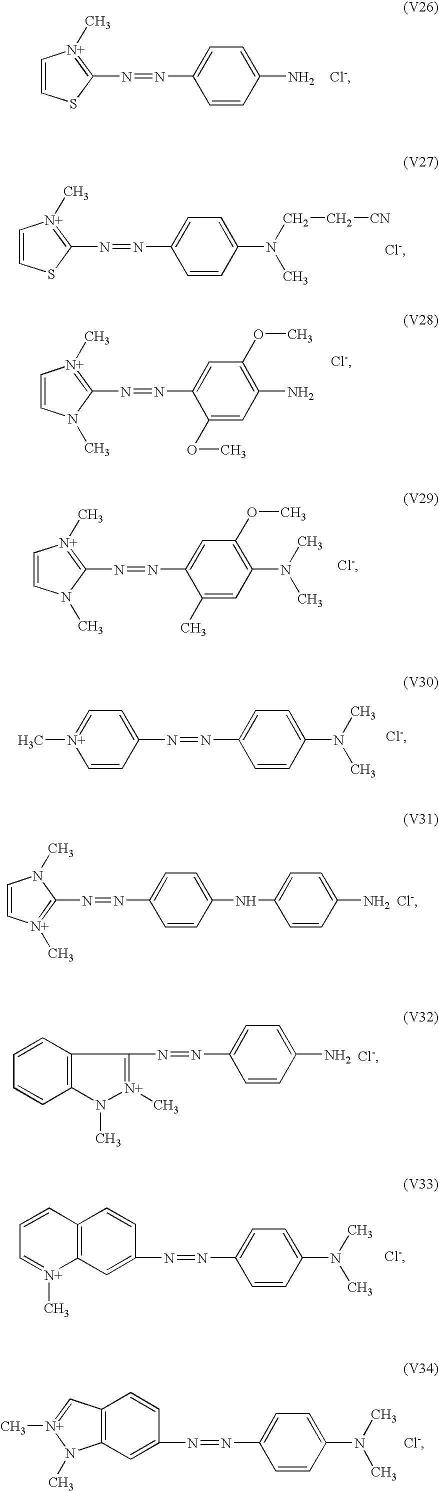

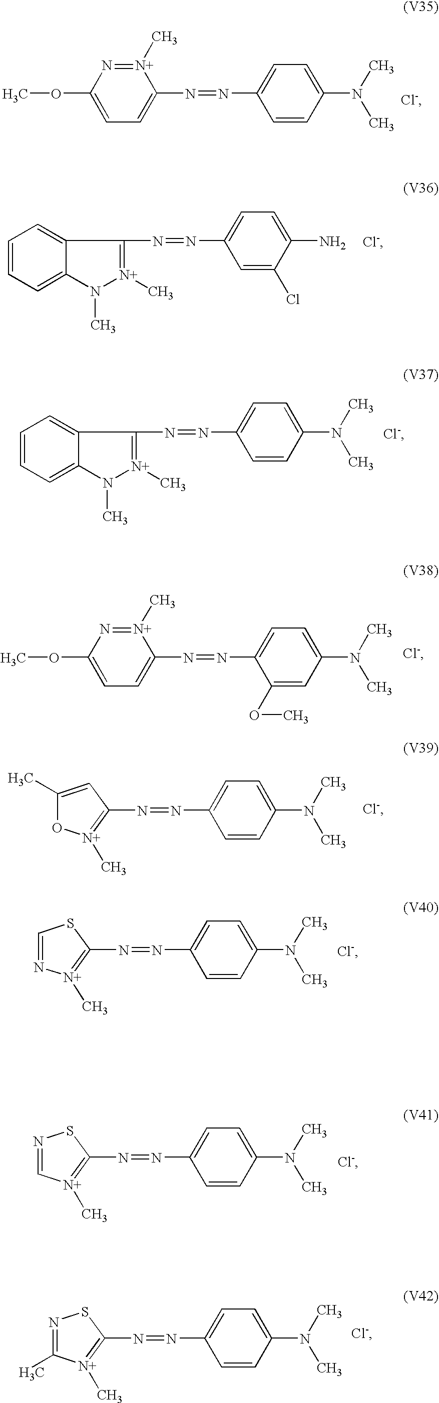

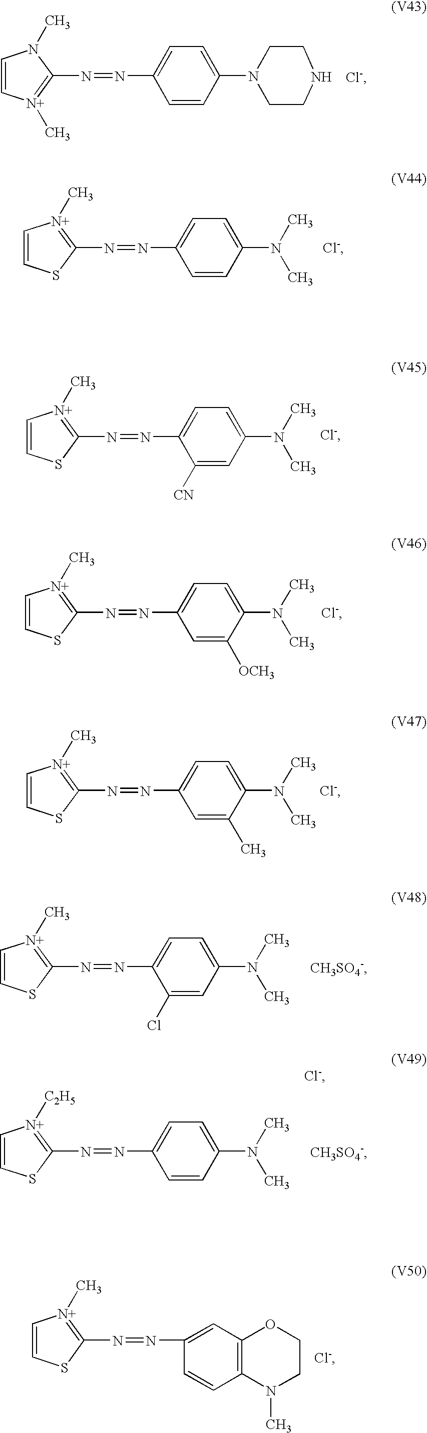

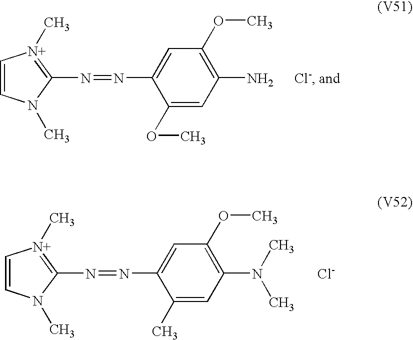

Among the compounds of structures (V1) to V52) described above, the compounds corresponding to the structures (V1), (V2), (V4), (V14) and (V31) are most particularly preferred.

Among the cationic direct dyes of formula (VI) which can be used in the ready-to-use dyeing compositions in accordance with the invention, there may be mentioned more particularly the compounds corresponding to the following structures (VII) to (VI12):

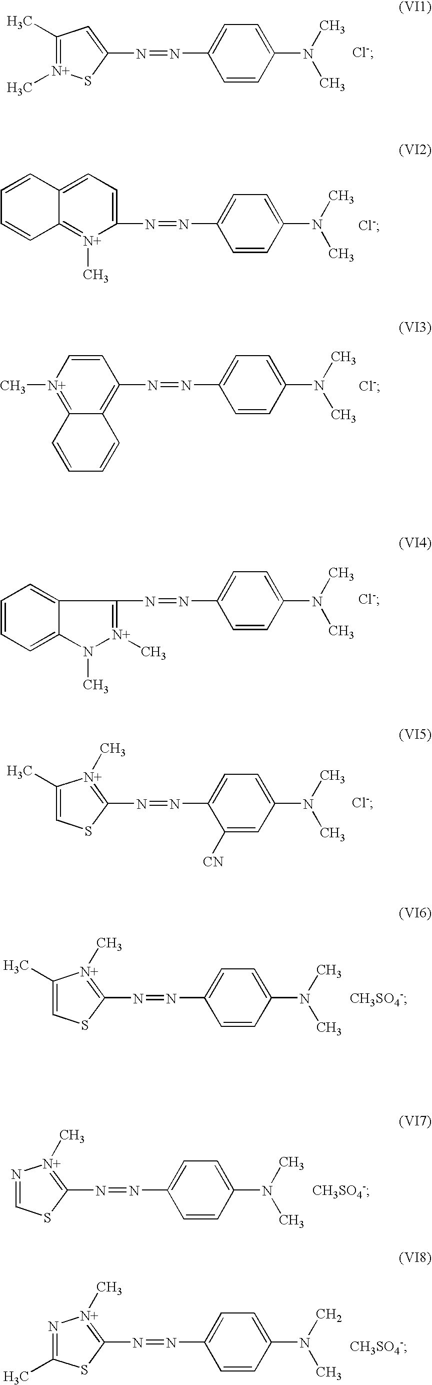

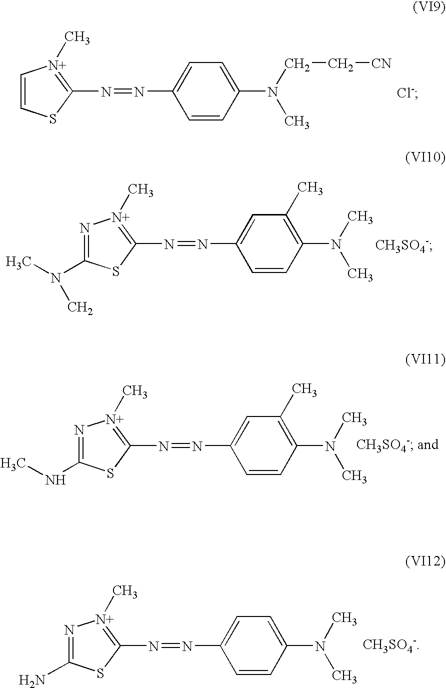

Among the cationic direct dyes of formula (VII) which can be used in the ready-to-use dyeing compositions in accordance with the invention, there may be mentioned more particularly the compounds corresponding to the following structures (VII1) to (VII18):

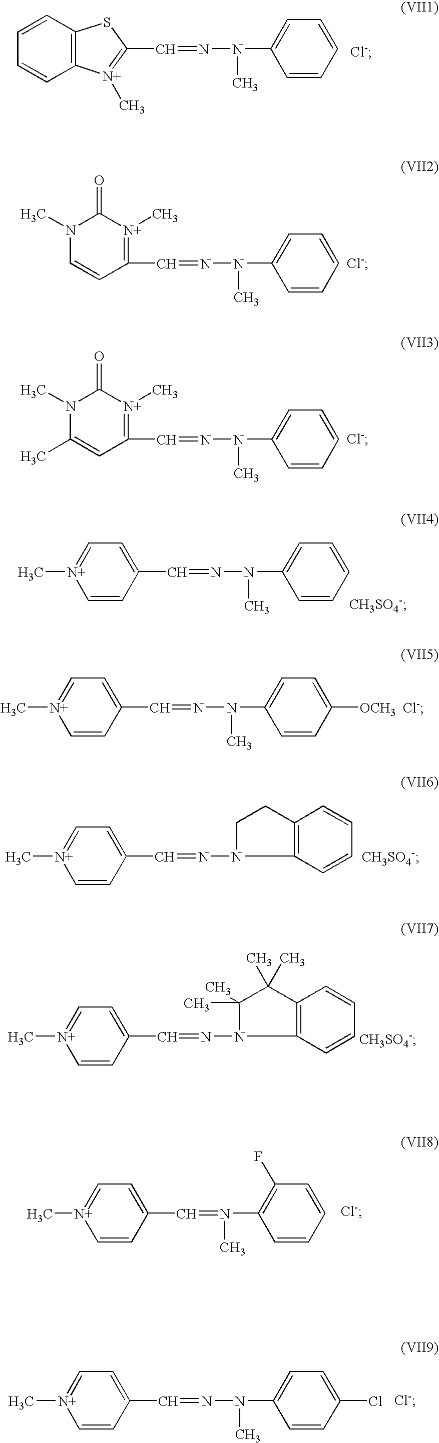

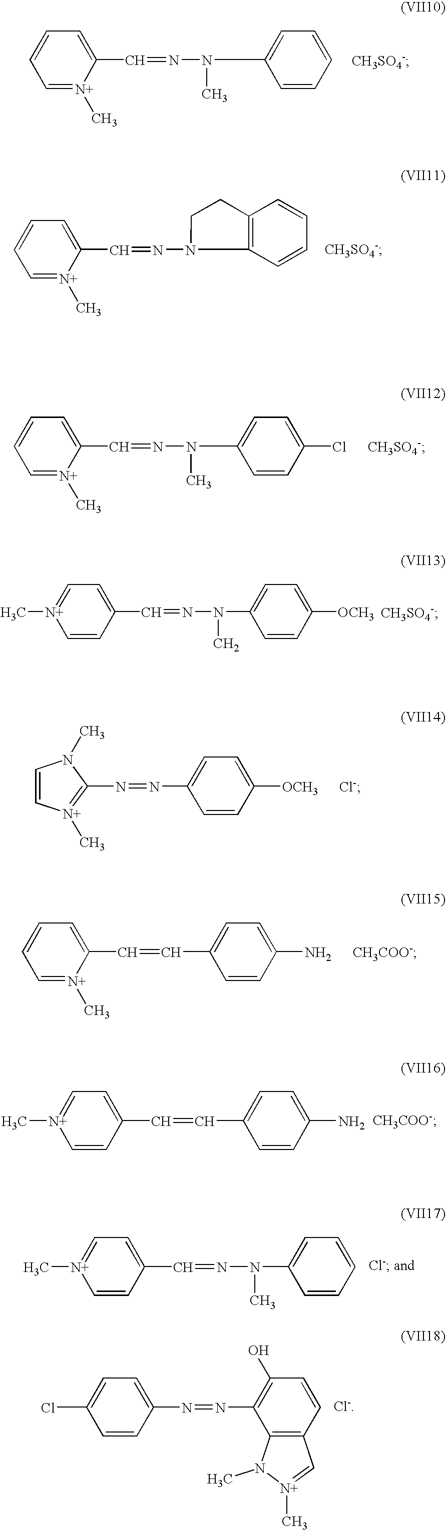

Among the particular compounds of structures (VII1) to (VII18) described above, the compounds corresponding to the structures (VII4), (VII5) and (VII13) are most particularly preferred.

Among the cationic direct dyes of formula (VII′) which can be used in the ready-to-use dyeing compositions in accordance with the invention, there may be mentioned more particularly the compounds corresponding to the following structures (VII′1) to (VII′3):

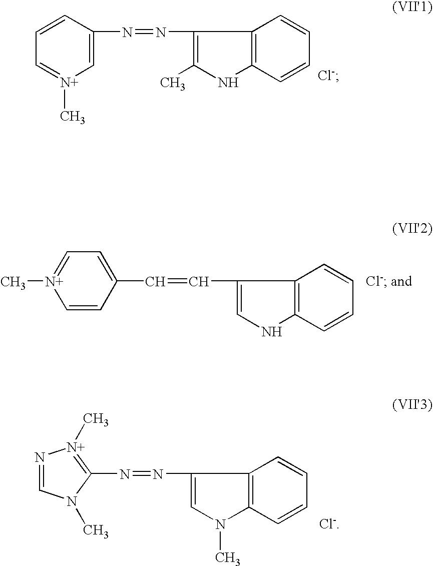

The cationic direct dye(s) which can be used according to the invention preferably represent from 0.001 to 10% by weight approximately of the total weight of the ready-to-use dyeing composition, and more preferably from 0.05 to 5% by weight approximately of this weight.

In general, the addition salts with an acid which can be used in the context of the dyeing compositions of the invention (oxidation bases and couplers) are in particular chosen from hydrochlorides, hydrobromides, sulphates and tartrates, lactates and acetates.

The medium appropriate for dyeing (or carrier) of the ready-to-use dyeing composition in accordance with the invention generally consists of water or of a mixture of water and of at least one organic solvent in order to solubilize the compounds which might not be sufficiently soluble in water. As organic solvent, there may be mentioned for example C₁-C₄ alkanols such as ethanol and isopropanol as well as aromatic alcohols such as benzyl alcohol, similar products and mixtures thereof.

The solvents may be present in proportions preferably of between 1 and 40% by weight approximately relative to the total weight of the dyeing composition, and still more preferably between 5 and 30% by weight approximately.

The pH of the ready-to-use composition in accordance with the invention is chosen such that the enzymatic activity of the laccase is sufficient. It is generally between 4 and 11 approximately, and preferably between 6 and 9 approximately.

The ready-to-use dyeing composition in accordance with the invention may also contain various adjuvants conventionally used in hair dyeing compositions, such as anionic, cationic, nonionic, amphoteric or zwitterionic surfactants or mixtures thereof, polymers, thickeners, antioxidants, enzymes different from the laccases used in accordance with the invention, such as for example peroxidases or oxido-reductases containing 2 electrons, penetrating agents, sequestering agents, perfumes, buffers, dispersing agents, film-forming agents, screening agents, vitamins, preservatives or opacifying agents.

Of course, persons skilled in the art will be careful to choose this or these optional additional compounds such that the advantageous properties intrinsically attached to the ready-to-use dyeing composition in accordance with the invention are not, or substantially not, impaired by the addition(s) envisaged.

The ready-to-use dyeing composition in accordance with the invention can be provided in various forms, such as in the form of liquids, creams, gels, optionally pressurized, or in any other form appropriate for dyeing keratinous fibres, in particular human hair. In this case, the oxidation dye(s) and the laccase(s) are present in the same ready-to-use composition, and consequently the said composition should be free of gaseous oxygen, so as to avoid any premature oxidation of the oxidation dye(s).

The subject of the invention is also a method of dyeing keratinous fibres, and in particular human keratinous fibres such as hair, using the ready-to-use dyeing composition as defined above.

According to this method, at least one ready-to-use dyeing composition as defined above is applied to the fibres for a sufficient time to develop the desired colour, after which they are rinsed, optionally washed with shampoo, rinsed again and dried.

The time necessary for the development of the colour on the keratinous fibres is generally between 3 and 60 minutes and still more precisely 5 and 40 minutes.

According to one particular embodiment of the invention, the method comprises a preliminary step consisting in storing in a separate form, on the one hand, a composition (A) comprising, in a medium appropriate for dyeing, at least one oxidation dye, at least one cationic direct dye and, on the other hand, a composition (B) containing, in a medium appropriate for dyeing, at least one enzyme of the laccase type, and then in mixing them at the time of use before applying this mixture to the keratinous fibres.

Another subject of the invention is a multi-compartment device or dyeing (kit) or any other multi-compartment packaging system in which a first comparment contains the composition (A) as defined above and a second compartment contains a composition (B) as defined above. These devices may be equipped with a means which makes it possible to deliver the desired mixture to the hair, such as the devices described in Patent FR-2,586,913 in the name of the applicant.

The following examples are intended to illustrate the invention without limiting the scope as a result.

### DYEING EXAMPLES 1 to 3

The following ready-to-use dyeing compositions were prepared (contents in grams):

| COMPOSITION | 1 | 2 | 3 |
|---|---|---|---|
| para-Phenylenediamine (oxidation | 0.550 | — | 0.283 |
| base) | | | |
| para-Aminophenol (oxidation | — | 0.147 | — |
| base) | | | |
| 5-N-(β-Hydroxyethyl) amino-2- | — | 0.165 | 0.283 |
| methylphenol (coupler) | | | |
| Red cationic direct dye of | 0.470 | — | — |
| structure (V1) | | | |
| Orange-coloured cationic direct | — | 0.051 | — |
| dye of structure (V4) | | | |
| Cationic direct dye: Basic Red | — | — | 0.094 |
| 76 (Arianor Madder Red) | | | |
| Laccase derived from Rhus | 1.8 | 1.8 | 1.8 |
| vernicifera laccase containing | | | |
| 180 units/mg sold by the company | | | |
| SIGMA | | | |
| Common dye carrier (*) | (*) | (*) | (*) |
| Demineralized water qs | 100 | 100 | 100 |

(*) : Common dye carrier:
Ethanol 20.0 g
(C₈-C₁₀)Alkyl polyglucoside in aqueous 4.8 g solution containing 60% of active (AS) substance (AS) sold under the name ORAMIX CG110 ® by the company SEPPIC
pH agent qs pH = 6.5

Each of the ready-to-use dyeing compositions described above was applied to locks of natural grey hair which is 90% white for 40 minutes at the temperature of 30° C. The hair was then rinsed, washed with a standard shampoo and then dried.

The hair was dyed in the shades presented in the table below:

| EXAMPLE | Shade obtained |
|---|---|
| 1 | Dark red |
| 2 | Very light red-copper |
| | blonde |
| 3 | Red-violet blonde |

In the examples described above, 1.8% of Rhus vernicifera laccase at 180 units/mg, sold by the company SIGMA, can be replaced by 1% of Pyricularia Orizae at 100 units/mg sold by the company I.C.N.

1-37. (canceled)

38. A composition comprising:
(a) at least one oxidation dye,
(b) at least one cationic direct dye, and
(c) at least one enzyme of the laccase type.

39. A composition according to claim 38, wherein said composition is used for treating keratinous fibres.

40. A composition according to claim 39, wherein said keratinous fibres are hair.

41. A composition according to claim 39, wherein said treating comprises oxidation dyeing of said keratinous fibres.

42. A composition according to claim 38, wherein said at least one enzyme of the laccase type is chosen from laccases of plant origin, animal origin, fungal origin, and bacterial origin and laccases obtained by biotechnology.

43. A composition according to claim 38, wherein said at least one enzyme of the laccase type is chosen from those produced by plants performing chlorophyll synthesis.

44. A composition according to claim 38, wherein said at least one enzyme of the laccase type is chosen from those extracted from plants chosen from Anacardiaceae, Podocarpaceae, Rosmarinus off., Solanum tuberosum, Iris sp., Coffea sp., Daucus carrota, Vinca minor, Persea americana, Catharenthus roseus, Musa sp., Malus pumila, Gingko biloba, Monotropa hypopithys, Aesculus sp., Acer pseudoplatanus, Prunus persica and Pistacia palaestina.

45. A composition according to claim 38, wherein said at least one enzyme of the laccase type is chosen from those derived from fungi chosen from Pyricularia orizae, Polyporus versicolor, Rhizoctonia praticola, Rhus vernicifera, Scytalidium, Polyporus pinsitus, Myceliophtora thermophila, Rhizoctonia solani, Tramates versicolor, Fomes fomentarius, Chaetomium thermophile, Neurospora crassa, Coriolus versicol, Botrytis cinerea, Rigidoporus lignosus, Phellinus noxius, Pleurotus ostreatus, Aspergillus nidulans, Podospora anserina, Agaricus bisporus, Ganoderma lucidum, Glomerella cingulata, Lactarius piperatus, Russula delica, Heterobasidion annosum, Thelephora terrestris, Cladosporium cladosporioides, Cerrena unicolor, Coriolus hirsutus, Ceriporiopsis subvermispora, Coprinus cinereus, Panaeolus papilionaceus, Panaeolus sphinctrinus, Schizophyllum commune, Dichomitius squalens and variants of all said fungi.

46. A composition according to claim 38, wherein said at least one enzyme of the laccase type is present in a quantity ranging from 0.5 to 2000 ulac units per 100 g of said composition.

47. A composition according to claim 38, wherein said at least one enzyme of the laccase type is present in a quantity ranging from 1000 to 4×10⁷ ulac units per 100 g of said composition.

48. A composition according to claim 38, wherein said at least one enzyme of the laccase type is present in a quantity ranging from 20 to 2×10⁶ ulac units per 100 g of said composition.

49. A composition according to claim 38, wherein said at least one oxidation dye is chosen from oxidation bases, couplers and the acid addition salts of all said at least one oxidation dyes.

50. A composition according to claim 49, wherein said acid addition salts are chosen from hydrochlorides, hydrobromides, sulphates, tartrates, lactates and acetates.

51. A composition according to claim 49, wherein said oxidation bases are chosen from para-phenylenediamines, double bases, para-aminophenols, ortho-aminophenols, heterocyclic bases and the acid addition salts of all said oxidation bases.

52. A composition according to claim 51, wherein said para-phenylenediamines are chosen from any of the compounds having the following formula (I) and any of their acid addition salts: 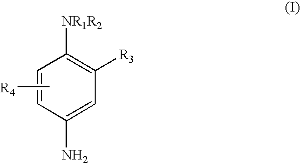
wherein:
R₁ is chosen from hydrogen groups, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups, C₁-C₄ alkyl groups substituted with at least one nitrogen-containing group, phenyl groups and 4′-aminophenyl groups;
R₂ is chosen from hydrogen groups, C₁-C₄ alkyl groups, monohydroxy(C₁-C₄ alkyl) groups, polyhydroxy(C₂-C₄ alkyl) groups, (C₁-C₄)alkoxy(C₁-C₄)alkyl groups and C₁-C₄ alkyl groups substituted with at least one nitrogen-containing group;
R₃ is chosen from hydrogen groups, halogen groups, C₁-C₄ alkyl groups, hydroxy(C₁-C₄ alkyl) groups, hydroxy(C₁-C₄ alkoxy) groups, acetylamino(C₁-C₄ alkoxy) groups, mesylamino(C₁-C₄alkoxy) groups and carbamoylamino(C₁-C₄ alkoxy) groups;
R₄ is chosen from hydrogen groups, halogen groups and C₁-C₄ alkyl groups.

53. A composition according to claim 52, wherein said para-phenylenediamines of formula (I) are chosen from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine and their acid addition salts.

54. A composition according to claim 53, wherein said para-phenylenediamines of formula (I) are chosen from para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-O-acetylaminoethyloxy-para-phenylenediamine and their acid addition salts.

55. A composition according to claim 51, wherein said double bases are chosen from compounds of the following formula (II), and their acid addition salts: 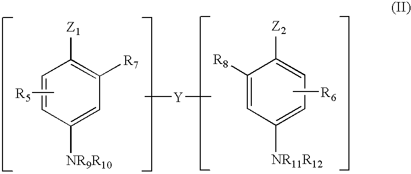
wherein:
Z₁ and Z₂, which may be identical or different, are each chosen from hydroxyl groups and —NH₂ groups each of which may optionally be substituted with a group chosen from C₁-C₄ alkyl groups and linking arms Y;
the linking arm Y is chosen from linear and branched, divalent alkylene groups comprising from 1 to 14 carbon atoms, which optionally may be interrupted by, or which optionally may end with, at least one nitrogen-containing group and/or at least one heteroatom and which optionally may be substituted with at least one group chosen from hydroxyl groups and C₁-C₆ alkoxy groups;
R₅ and R₆, which may be identical or different, are each chosen from hydrogen groups, halogen groups, C₁-C₄ alkyl groups, monohydroxy(C₁-C₄ alkyl) groups, polyhydroxy(C₂-C₄ alkyl) groups, amino(C₁-C₄ alkyl) groups and linking arms Y;
R₇, R₈, R₉, R₁₀, R₁₁ and R₁₂, which may be identical or different, are each chosen from hydrogen groups, linking arms Y and C₁-C₄ alkyl groups;
it being understood that said compounds of formula (II) contain only one linking arm Y per molecule.

56. A composition according to claim 55, wherein said heteroatoms are chosen from oxygen, sulphur and nitrogen.

57. A composition according to claim 51, wherein said double bases are chosen from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane and their acid addition salts.

58. A composition according to claim 57, wherein said double bases of formula (II) are chosen from N,N′-bis(β-hydroxyethyl)-N,N′-bis(4′-aminophenyl)-1,3-diaminopropanol, 1,8-bis(2,5-diaminophenoxy)-3,5-dioxaoctane and their acid addition salts.

59. A composition according to claim 51, wherein said para-aminophenols are chosen from compounds corresponding to the formula (III) and their acid addition salts: 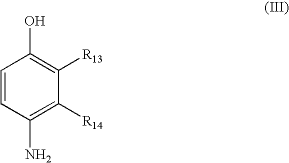
wherein:
R₁₃ is chosen from hydrogen groups, halogen groups, C₁-C₄ alkyl groups, hydroxy(C₁-C₄ alkyl) groups, (C₁-C₄)alkoxy(C₁-C₄)alkyl groups, amino(C₁-C₄)alkylamino(C₁-C₄ alkyl) groups, and hydroxy(C₁-C₄)alkylamino(C₁-C₄ alkyl) groups;
R₁₄ is chosen from hydrogen groups, halogen groups, C₁-C₄ alkyl groups, monohydroxy(C₁-C₄ alkyl) groups, polyhydroxy(C₂-C₄ alkyl) groups, amino(C₁-C₄ alkyl) groups, cyano(C₁-C₄ alkyl)(C₁-C₄)alkyl groups and (C₁-C₄)alkoxy(C₁-C₄)alkyl groups;
it being understood that at least one of the groups chosen from R₁₃ and R₁₄ is hydrogen.

60. A composition according to claim 59, wherein said para-aminophenols of formula (III) are chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluoro-phenol and their acid addition salts.

61. A composition according to claim 51, wherein said ortho-aminophenols are chosen from 2-aminophenol, 2-amino-5-methylphenol, 2-amino-6-methylphenol, 5-acetamido-2-aminophenol and their acid addition salts.

62. A composition according to claim 51, wherein said heterocyclic bases are chosen from pyridine derivatives, pyrimidine derivatives, pyrazole derivatives, pyrazolopyrimidine derivatives and their acid addition salts.

63. A composition according to claim 49, wherein said at least one oxidation base is present in said composition in an amount ranging from 0.0005% to 12% by weight of the total weight of said composition.

64. A composition according to claim 63, wherein said at least one oxidation base is present in said composition in an amount ranging from 0.005% to 6% by weight of the total weight of said composition.

65. A composition according to claim 49, wherein said couplers are chosen from meta-phenylenediamines, meta-aminophenols, meta-diphenols, heterocyclic couplers and their acid addition salts.

66. A composition according to claim 65, wherein said couplers are chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, β-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole, 6-methylpyrazolo-[1,5-a]benzimidazole and their acid addition salts.

67. A composition according to claim 49, wherein said couplers are present in said composition in an amount ranging from 0.0001% to 10% by weight of the total weight of said composition.

68. A composition according to claim 67, wherein said couplers are present in said composition in an amount ranging from 0.005% to 5% by weight of the total weight of said composition.

69. A composition according to claim 38, wherein said at least one cationic direct dye is chosen from cationic aminoanthraquinones, cationic mono-azo compounds, diazo compounds and cationic naphthoquinones.

70. A composition according to claim 69, wherein said at least one cationic direct dye is chosen from [8-[(p-aminophenyl)azol]-7-hydroxy-2-naphthyl]trimethylammonium chloride, 3-[(4-amino-6-bromo-5,8-dihydro-1-hydroxy-8-imino-5-oxo-2-naphthalenyl)amino]-N,N,N-trimethylbenzenaminium chloride, 7-hydroxy-8-[(2-methoxyphenyl)azo]-N,N,N-trimethyl-2-naphthalenaminium chloride, [8-[(4-amino-2-nitrophenyl)azo]-7-hydroxy-2-naphthyl]trimethylammonium chloride and 3-[(4,5-dihydro-3-methyl-5-oxo-1-phenyl-1H-pyrazol-4-yl)azo]-N,N, N-trimethylbenzenaminium chloride.

71. A composition according to claim 38, wherein said at least one cationic direct dye is chosen from:
a) compounds having the following formula (V): 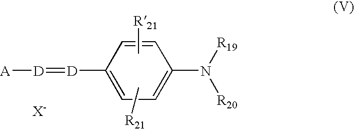
wherein:
D, which may be identical or different, are each chosen from nitrogen atoms and —CH groups;
R₁₉ and R₂₀, which may be identical or different, are each chosen from hydrogen groups, 4′-aminophenyl groups and C₁-C₄ alkyl groups which optionally may be substituted with at least one group chosen from —CN, —OH and —NH₂ groups, or each R₁₉ and R₂₀ may optionally form a ring which includes a carbon atom of the benzene ring which ring formed optionally may contain at least one atom chosen from oxygen and nitrogen, and which ring formed optionally may be substituted with at least one group chosen from C₁-C₄ alkyl groups;
R₂, and R₁₂₁, which may be identical or different, are each chosen from hydrogen groups, halogen groups, cyano groups, C₁-C₄ alkoxy groups and acetyloxy groups;
X⁻ represents an anion chosen from chloride, methyl sulphate and acetate;
A represents a group chosen from compounds having the following structures A1 to A19: 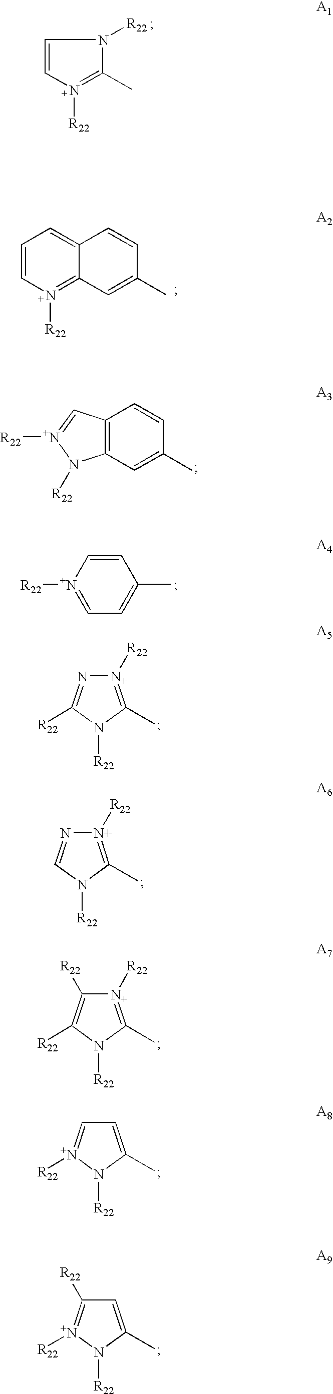 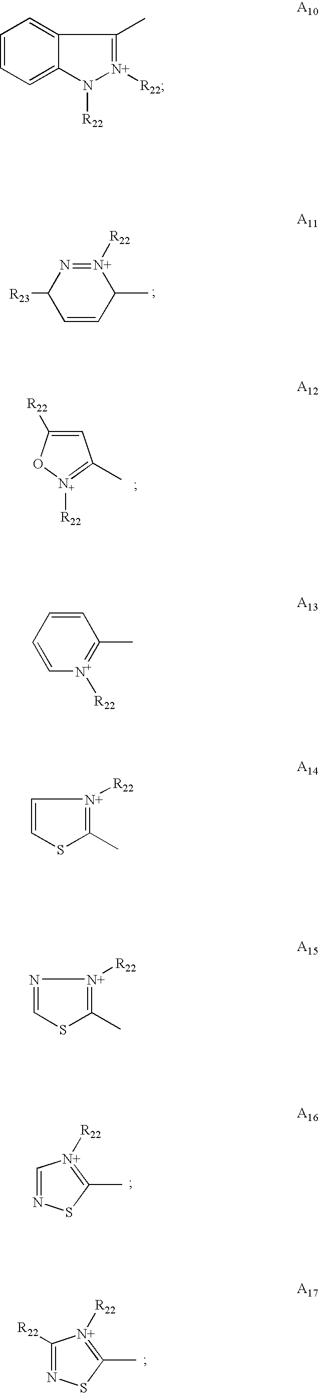 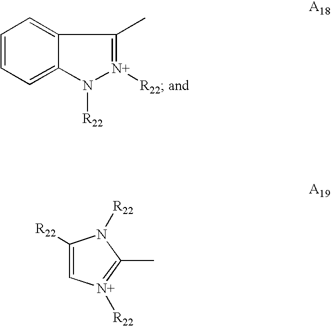
wherein
R₂₂, which may be identical or different, are each chosen from C₁-C₄ alkyl groups which optionally may be substituted with hydroxyl groups; and
R₂₃ is chosen from C₁-C₄ alkoxy groups.
b) compounds having the following formula (VI): 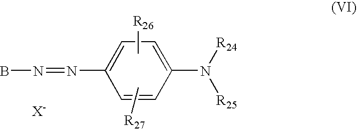
wherein:
R₂₄ is chosen from hydrogen groups and C₁-C₄ alkyl groups;
R₂₅ is chosen from hydrogen groups, alkyl groups which optionally may be substituted with a group chosen from —CN groups and amino groups, and 4′-aminophenyl groups, or R₂₅ optionally may form a ring with R₂₄ which ring formed optionally may contain at least one heteroatom chosen from oxygen and nitrogen and which ring formed optionally may be substituted with C₁-C₄ alkyl groups;
R₂₆ and R₂₇, which may be identical or different, are each chosen from hydrogen groups, halogen groups chosen from bromine, chlorine, iodine and fluorine, C₁-C₄ alkyl groups, C₁-C₄ alkoxy groups and —CN groups;
X⁻ is chosen from anions chosen from chloride, methyl sulphate and acetate; and
B is chosen from groups having the following structures B1 to B6: 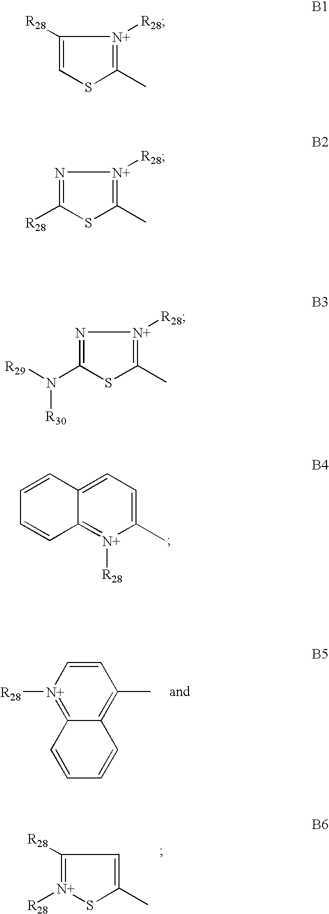
wherein R₂₈, which may be identical or different, are each chosen from C₁-C₄ alkyl groups; and
R₂₉ and R₃₀, which may be identical or different, are each chosen from hydrogen groups and C₁-C₄ alkyl groups.
c) compounds having the following formulae (VII) and (VII′): 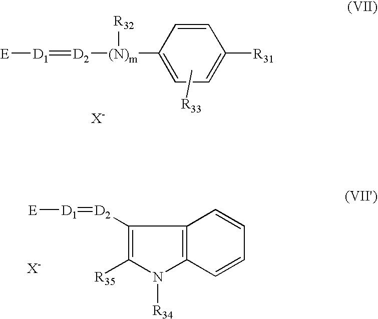
wherein:
R₃₁ is chosen from hydrogen groups, C₁-C₄ alkoxy groups, halogen groups chosen from bromine, chlorine, iodine and fluorine, and amino groups;
R₃₂ is chosen from hydrogen groups and C₁-C₄ alkyl groups, or R₃₂ optionally may form a ring with a carbon atom of the benzene ring and which ring formed optionally may contain oxygen and which ring formed optionally may be substituted with at least one C₁-C₄ alkyl group;
R₃₃ is chosen from hydrogen groups and halogen groups chosen from bromine, chlorine, iodine and fluorine;
R₃₄ and R₃₅, which may be identical or different, are each chosen from hydrogen groups and C₁-C₄ alkyl groups;
D₁ and D₂, which may be identical or different, are each chosen from nitrogen atoms and —CH groups;
m equals 0 or 1;
it being understood that when R₃₁ is chosen from unsubstituted amino groups, simultaneously both D₁ and D₂ are chosen from —CH groups, and m=0;
X⁻ is chosen from chloride, methyl sulphate and acetate; and
E is chosen from the following structures E1 to E8: 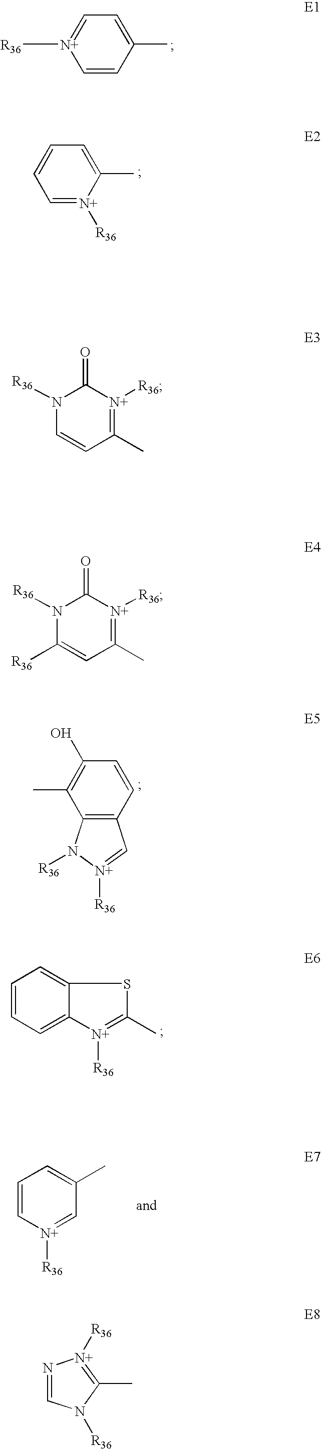
wherein
R₃₆, which may be identical or different, are each chosen from C₁-C₄ alkyl groups; it being understood that when both m equals 0 and D₁ is chosen from nitrogen atoms, E optionally may also be chosen from the following structure E9: 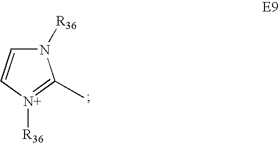
wherein R₃₆, which may be identical or different, are each chosen from C₁-C₄ alkyl groups.

72. A composition according to claim 71, wherein said at least one cationic direct dye of formula (V) is chosen from compounds having the following structures (V1) to (V52):

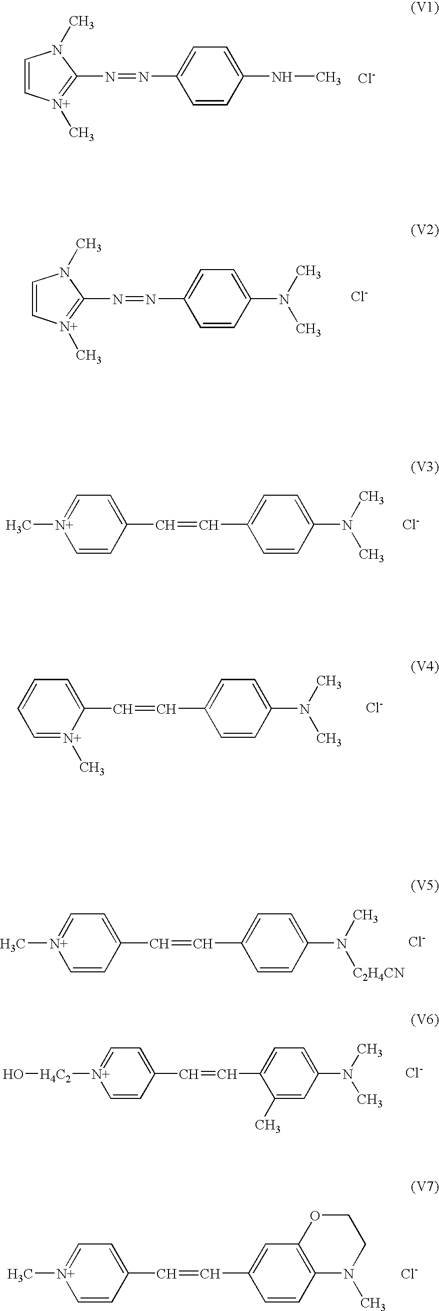

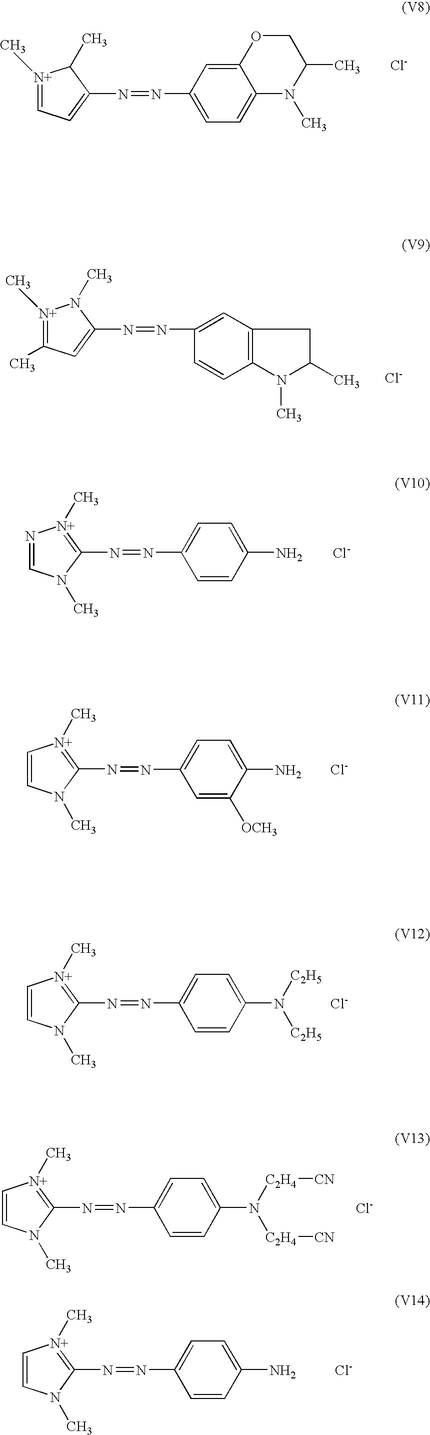

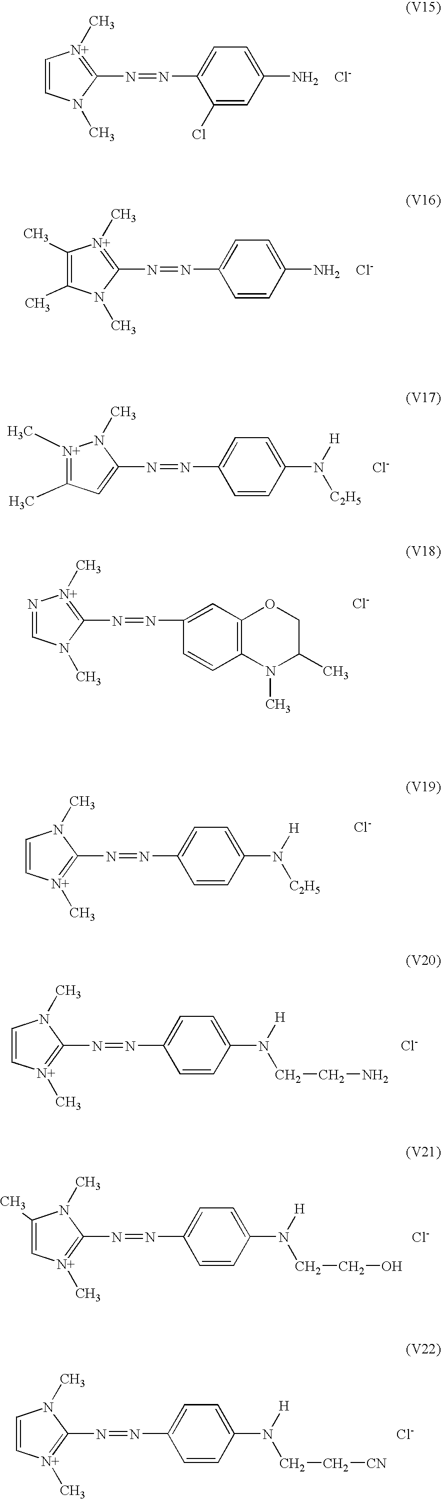

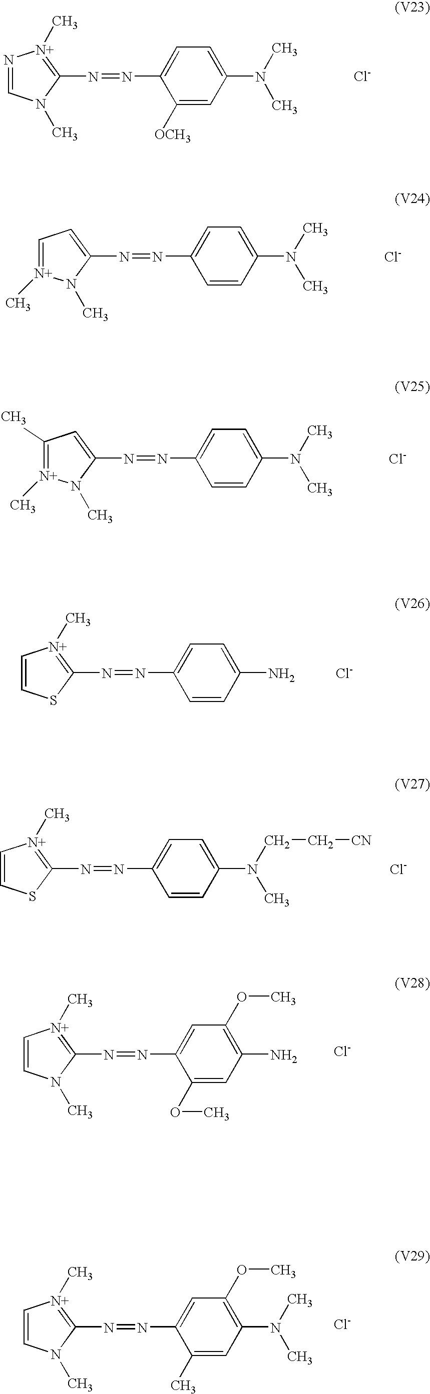

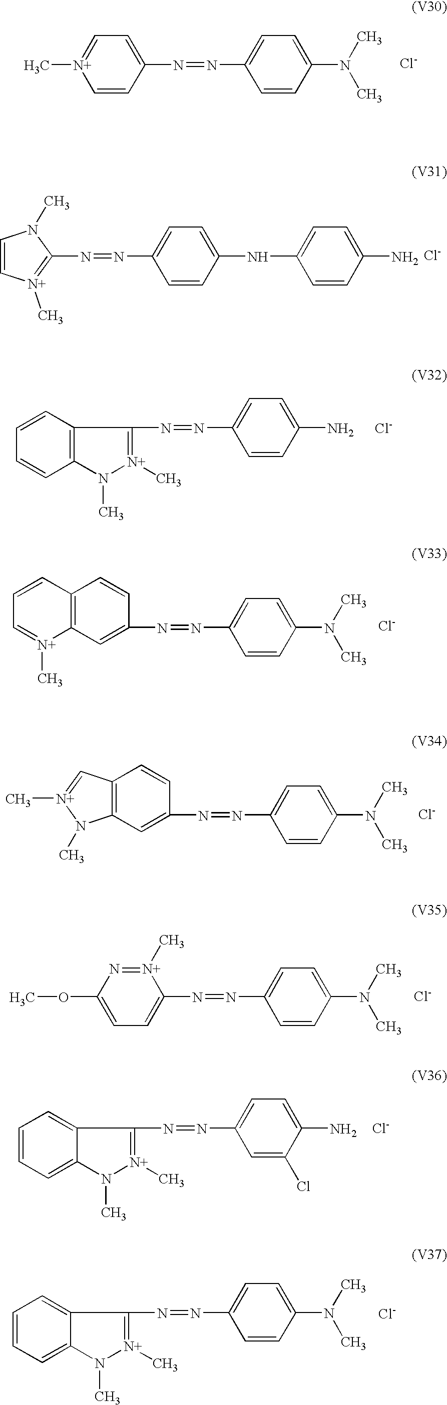

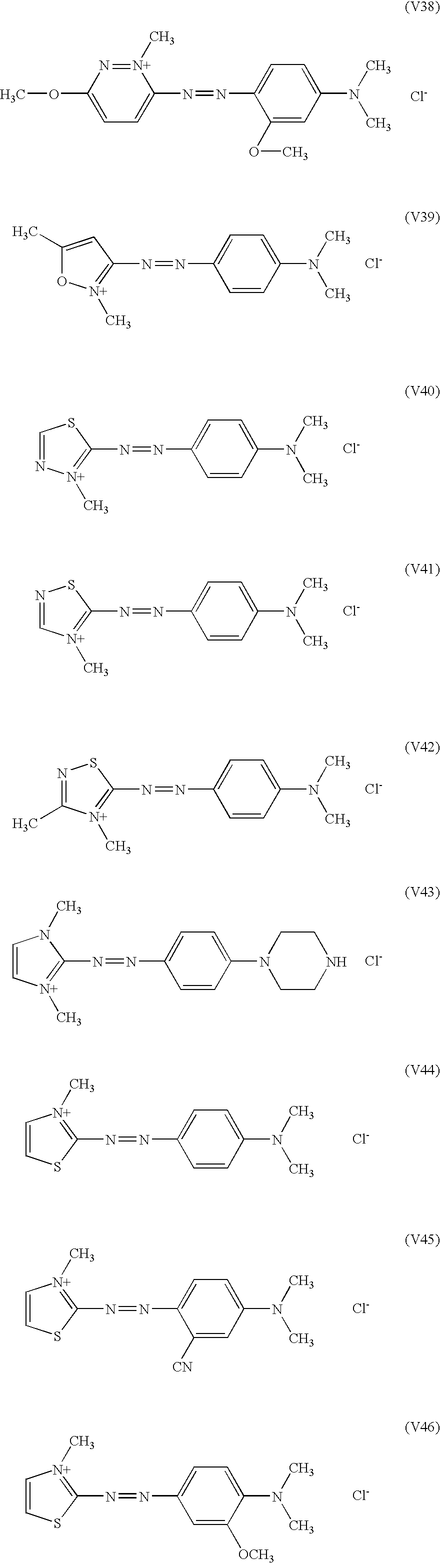

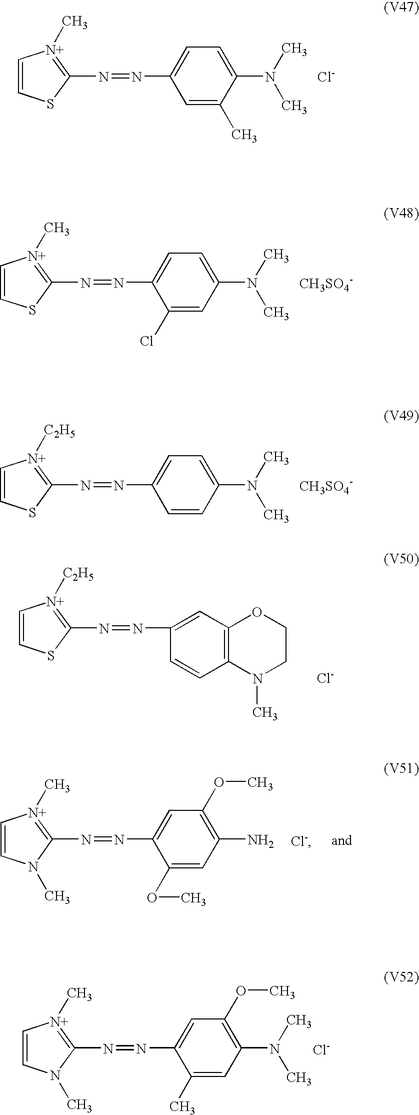

73. A composition according to claim 38, wherein said at least one cationic direct dye is chosen from the following structures (VI1) to (VI12):

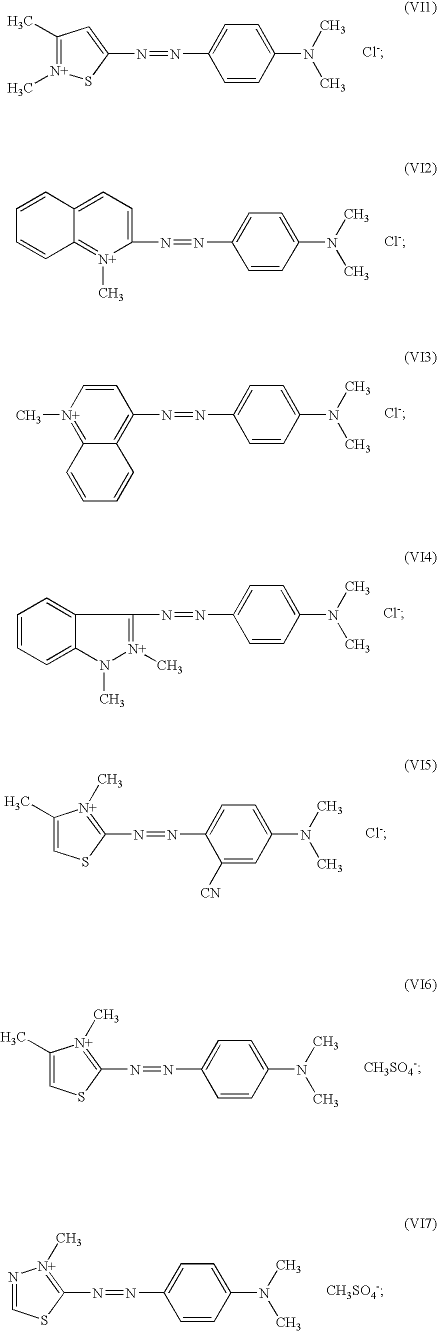

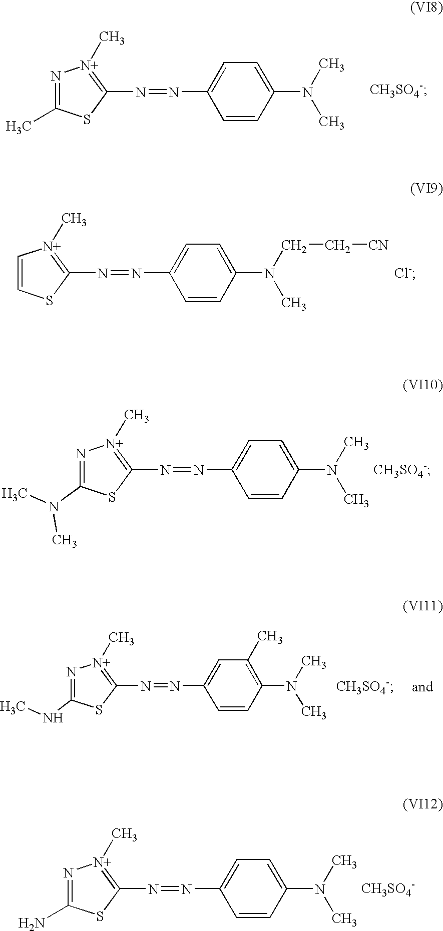

74. A composition according to claim 71, wheren said at least one cationic direct dye of formula (VII) is chosen from compounds of the following structures (VII1) to (VII18):

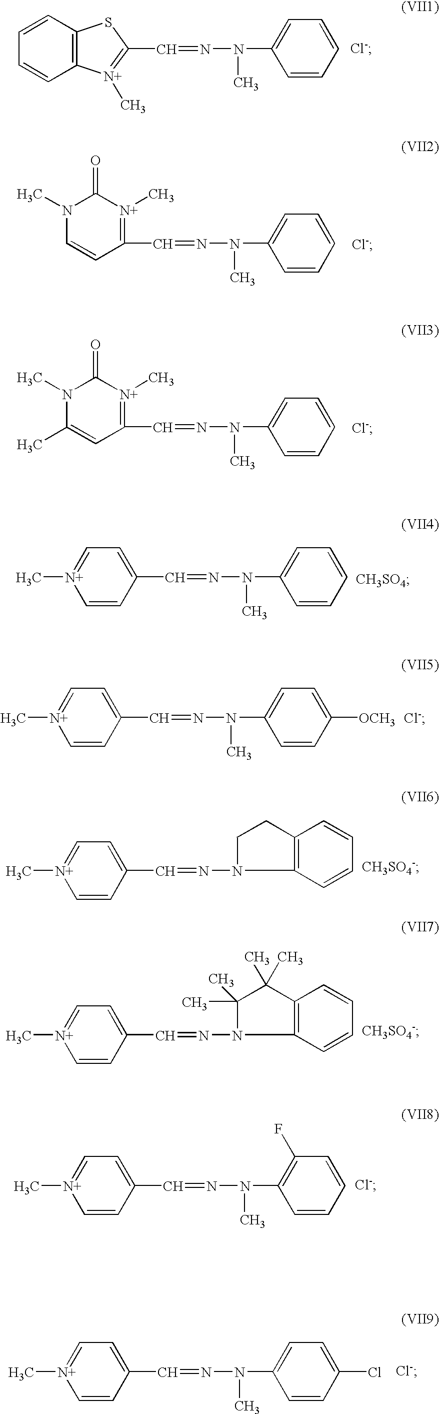

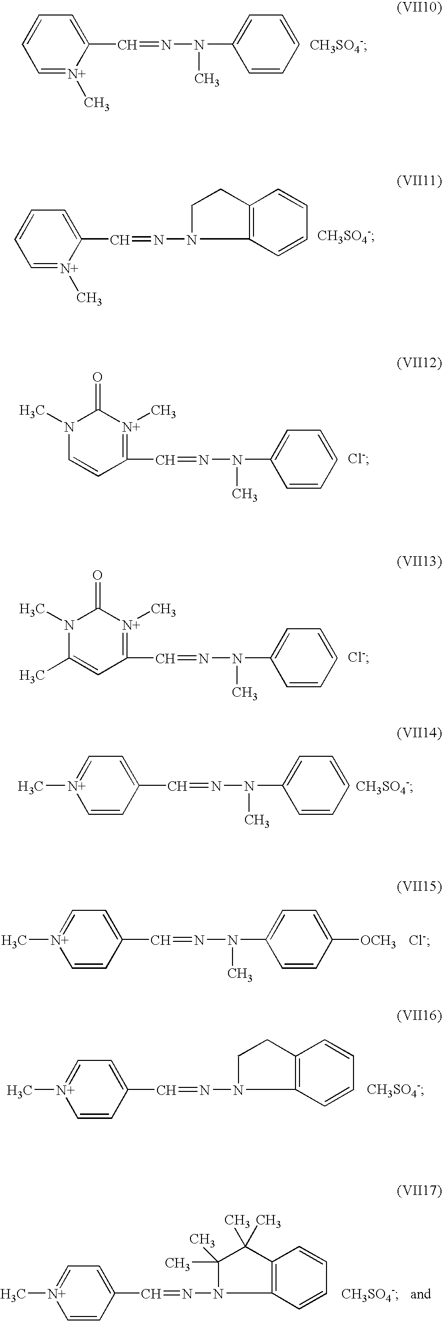

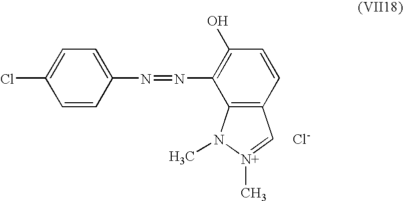

75. A composition according to claim 71, wherein said at least one cationic direct dye of formula (VII′) is chosen from compounds having the following structures (VII′1) to (VII′3):

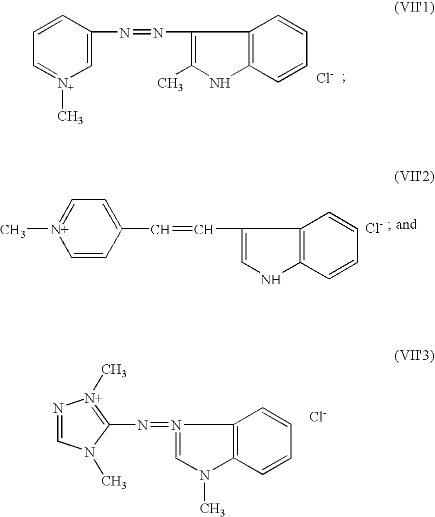

76. A composition according to claim 38, wherein said at least one cationic direct dye is present in said composition in an amount ranging from 0.001% to 10% by weight of the total weight of said composition.

77. A composition according to claim 76, wherein said at least one cationic direct dye is present in said composition in an amount ranging from 0.05% to 5% by weight of the total weight of said composition.

78. A composition according to claim 38, further comprising a medium appropriate for keratinous fibres.

79. A composition according to claim 78, wherein said medium appropriate for keratinous fibres is chosen from water and at least one organic solvent.

80. A composition according to claim 79, wherein said at least one organic solvent is chosen from (C₁-C₄ alkyl)alcohols and aromatic alcohols.

81. A composition according to claim 78, wherein said medium appropriate for keratinous fibres is present in said composition in an amount ranging from 1% to 40% by weight relative to the total weight of said composition.

82. A composition according to claim 81, wherein said medium appropriate for keratinous fibres is present in said composition in an amount ranging from 5% to 30% by weight relative to the total weight of said composition.

83. A composition according to claim 38 having a pH in a range from 4 to 11.

84. A composition according to claim 83 having a pH in a range from 6 to 9.

85. A composition according to claim 38, further comprising at least one suitable adjuvant chosen from anionic, cationic, nonionic, amphoteric and zwitterionic surfactants, cationic, nonionic, amphoteric and zwitterionic polymers, thickeners, antioxidants, enzymes different from said at least one enzyme of the laccase type defined in claim 38, penetrating agents, sequestering agents, perfumes, buffers, dispersing agents, film-forming agents, screening agents, vitamins, preservatives and opacifiers.

86. A composition according to claim 38, wherein said composition is a ready-to-use composition.

87. A composition according to claim 38 in the form of a liquid, a cream, a gel, or in any other form suitable for keratinous fibres.

88. A composition according to claim 87, wherein said composition form may optionally be pressurized.

89. A method for dyeing keratinous fibres comprising the step of applying a ready-to-use composition to said fibres for a time sufficient to achieve a desired colouration, wherein said ready-to-use composition comprises:
(a) at least one oxidation dye,
(b) at least one cationic direct dye, and
(c) at least one enzyme of the laccase type.

90. A method according to claim 89, further comprising the step of rinsing said composition from said fibres.

91. A method according to claim 90, further comprising the step of washing the fibres.

92. A method according to claim 91, further comprising the step of rinsing said fibres a second time.

93. A method according to claim 92, further comprising the step of drying said fibres.

94. A method according to claim 89, wherein said time sufficient to achieve a desired colouration ranges from 3 to 60 minutes.

95. A method according to claim 94, wheren said time sufficient to achieve a desired colouration ranges from 5 to 40 minutes.

96. A method for dyeing keratinous fibres comprising the steps of:
(a) storing a first composition,
(b) storign a second composition separately from said first composition,
(c) mixing the first composition with the second composition to form a mixture, and
(d) applying said mixture to said keratinous fibres for a time sufficient to achieve a desired colouration,
wherein said first composition comprises at least one oxidation dye and at least one cationic direct dye, in a medium suitable for keratinous fibres, and
wherein said second composition comprises at least one enzyme of the laccase type in a medium suitable for keratinous fibres.

97. A multicompartment device or dyeing kit, wherein said device or kit comprises:
(a) a first compartment comprising a first composition, and
(b) a second compartment comprising a second composition,
wherein said first compartment comprises at least one oxidation dye, and at least one cationic direct dye, in a medium suitable for keratinous fibres, and
wherein said second compartment comprises at least one enzyme of the laccase type in a medium suitable for keratinous fibres.
